(19) United States
(12) Patent Application Publication
(10) Pub. No.: US 2015/0176977 A1
(43) Pub. Date: Jun. 25, 2015

(54) METHODS AND DEVICES FOR DETERMINING POSITION OR DISTANCE

(71) Applicant: Lemoptix SA, Lausanne (CH)

(72) Inventors: Nicolas Abele, Lausanne (CH); Jonathan Masson, Lausanne (CH); Lucio Kilcher, Montreux (CH)

(21) Appl. No.: 14/136,475

(22) Filed: Dec. 20, 2013

(51) Int. Cl. G01B 11/14; A61B 3/00; G02B 26/08

(52) U.S. Cl. CPC G01B 11/14; G02B 26/0833; A61B 3/0016

(57) ABSTRACT

A method for detecting the positioning of an entity, comprising the steps of (a) using a projector, which comprises a laser and a MEMS micro mirror arranged to receive light from the laser and which can oscillate about at least one oscillation axis, to project light towards the entity to project an image which is composed of pixels onto the entity, wherein the image has a first density of pixels; (b) changing the density of pixels in the projected image; (c) sensing at least a portion of light of the projected image which is reflected from the entity; and (d) using the sensed portion of light to determine the position of the entity. There is also provided a corresponding device for detecting position. There is also provided a method and device for determining distance which involves using the sensed portion of light to determine the distance between the entity and projector.

### FIELD OF THE INVENTION

The present invention concerns method and devices for detecting position or distance, and in particular to method and devices for detecting position or distance which involve changing the density of pixels in a projected image to provide for a more efficient position and/or distance measuring device and method.

### DESCRIPTION OF RELATED ART

Distance measurement devices which comprise a light source which directs light to an entity and a sensor which senses light which has been reflected from that entity to determine the distance between the light source and entity, are known in the art. Typically the sensors of these distance measurement devices can determine the distance by determining the time of flight of the light or the phase of the light which it receives, or by triangulation using multiple cameras. Typically the light sources of these distance measurement devices are configured to emit infrared light or may emit visible light; the light sources are further usually configured so that the light which they emit produces a pattern of pixels (either rounded pixels, or line pixels) on the entity.

However in many cases the light source provides light which illuminates a regions which is larger than the entity; as a result the sensor not only receives light which is reflected by the entity but also light which is reflected by other objects which are near the entity. In cases where the only the distance to the entity is of interest, the sensor uses unnecessary power and resources in processing the light which is reflected by other objects which are near the entity.

Devices which can determine the position of an entity also known. These devices work on the same principle as the distance measurement devices: the distance to the entity is measured in the same way, and the position of the entity, within an area defined by the light emitted from the light source, can be determined based on measured distance to the entity.

It is an objective of the present invention to obviate or mitigate some of the above-mentioned disadvantages.

### BRIEF SUMMARY OF THE INVENTION

According to the invention, these aims are achieved by means of a method for detecting the positioning of an entity, comprising the steps of (a) using a projector, which comprises a laser and a MEMS micro minor arranged to receive light from the laser and which can oscillate about at least one oscillation axis, to project light towards the entity to project an image which is composed of pixels onto the entity, wherein the image has a first density of pixels; (b) changing the density of pixels in the projected image; (c) sensing at least a portion of light of the projected image which is reflected from the entity; and (d) using the sensed portion of light to determine the position of the entity.

The projected light may comprise discrete light beams each of which defines a respective pixel; and wherein the method may comprise, receiving the discrete light beams which are reflected by the entity using a sensor which comprises a matrix of discrete light detectors; determining the position of the entity based on which of the discrete light detectors receive the reflected discrete light beams.

The entity may be a human body. The entity may be a body part. The entity may be the pupil of an eye of a person.

The method may further comprise determining the direction in which the person is looking from the determined position of the pupil of the eye.

The method may further comprise repeating steps (c) and (d) a plurality of times to obtain a plurality of determined positions of the entity, and using the plurality of determined positions to determine movement of the entity.

The projected light comprises discrete light beams each of which defines a respective pixel; and the method may comprise, at a first time instance, receiving discrete light beams which are reflected by the entity using a sensor which comprises a matrix of discrete light detectors and then determining a first position of the entity based on which of the discrete light detectors receive the reflected discrete light beams; and at a second time instance, receiving discrete light beams which are reflected by the entity using a sensor which comprises a matrix of discrete light detectors and then determining a second position of the entity based on which of the discrete light detectors receive the reflected discrete light beams; using the determined first and second positions to determine movement of the entity.

The step of using the determined first and second positions to determine movement of the entity may comprise subtracting the first position from the second position to determine movement of the entity.

The method may further comprise the steps of, identifying an area of interest within the image; and wherein the step of changing the density of pixels in the projected image comprises increasing the density of pixels in the area of interest only.

The area of interest may be an area of the image which is projected onto a predefined body part of the person.

The area of interest may be an area of the image which is projected onto an eye of the person.

The step of changing the density of pixels in the projected image may further comprise, decreasing the density of pixels outside of the area of interest.

The pixels may be configured to be spot-shaped and/or line-shaped and/or elliptical shaped.

The step of changing the density of pixels in the projected image may comprise, changing the laser modulation speed. The laser modulation speed is the speed at which the laser outputs consecutive light beams, each of the consecutive light beams defining an independent pixel of the image.

The step of changing the density of pixels in the projected image may comprise, changing the speed at which the MEMS micro mirror oscillates about its at least one oscillation axis and/or changing the amplitude of oscillation of the MEMS micro mirror about its at least one oscillation axis.

The pixels of the projected image may be arranged in a predefined pattern, and wherein the step of sensing at least a portion of light of the projected image which is reflected from the entity may comprise sensing light using a sensor which comprises light detectors which are arranged in a pattern equal to the predefined pattern of the pixels of the projected image.

The step of using a projector to project light towards the entity to project an image which is composed of pixels onto the entity, may comprise, emitting light from the laser; receiving the light at the MEMS micro mirror and directing the light to the entity using the MEMS micro minor; oscillating the MEMS micro minor about two orthogonal oscillation axes to scan the light across the entity to project an image which is composed of pixels onto the entity.

The step of using a projector to project light towards the entity to project an image which is composed of pixels onto the entity, may comprise, emitting light from the laser; passing the light through a first lens; passing the light through a second lens; and directing the light towards the entity using the MEMS micro minor.

The first and/or second lens may be one of, a semi-cylindrical lens; a cylindrical lens; an planoconvex semi-cylindrical lens; a biconvex cylindrical lens, concavoconvex cylindrical lens as, a planoconcave semi-cylindrical lens, a biconcave cylindrical lens, or a convexoconcave cylindrical lens.

Preferably, the step of using a projector to project light towards the entity to project an image which is composed of pixels onto the entity, may comprise, emitting light from the laser; passing the light through a first semi-cylindrical lens; passing the light through a second semi cylindrical lens; and directing the light towards the entity using the MEMS micro minor.

The first lens may be configured to collimate the beam in a first axis only and let the light diverging in a second axis which is orthogonal to the first axis, and wherein the second lens may be configured to focus the light along the second axis.

The first lens and the second lens are arranged so that the longest axis of each the lenses are perpendicular to each other. Preferably the first semi-cylindrical lens and the second semi-cylindrical lens are arranged so that the longest axis of each the semi-cylindrical lenses are perpendicular to each other. Preferably, each of the first semi-cylindrical lens and the second semi-cylindrical lens comprise a curved surface and a flat surface, and the first semi-cylindrical lens and the second semi-cylindrical lens are arranged so that the curved surface of the first semi-cylindrical lens is closest to the flat surface of the second semi-cylindrical lens.

Preferably the order of the steps are first the light is emitted from the laser, then the light is passed through the first lens, then the light is passed through the second lens, and then the light is directed by the MEMS micro mirror towards the entity.

The step of using a projector to project light towards the entity to project an image which is composed of pixels onto the entity may comprise, emitting light from the laser; passing the light through a collimating lens; reflected onto the MEMS micro minor; passing the light through a second lens; The collimating lens may comprise a spherical or aspherical lens.

The collimating lens may be configured to collimate the light beam in a first and second axis, wherein the first and second axes are orthogonal; and the second lens may be configured to focus light along a third axis. Preferably the third axis is orthogonal to the first and second axes. The collimating lens may be configured to collimate the light beam in a first and second axis, wherein the first and second axes are orthogonal; and the second lens may be configured to focus or diverge light along a third axis which is orthogonal to the first and second axes. It will be understood that the second lens may be configured to focus or diverge the light in any axis, and preferably is configured to focus or diverge light in an axis which is parallel to the axis of oscillation of the MEMS minor. In other words preferably the third axis is parallel to the axis of oscillation of the MEMS minor.

The second lens may be one of, a semi-cylindrical lens; a cylindrical lens; a planoconvex semi-cylindrical lens; a biconvex cylindrical lens, concavoconvex cylindrical lens as, a planoconcave semi-cylindrical lens, a biconcave cylindrical lens, or a convexoconcave cylindrical lens.

Preferably the order of the steps are, first the light is emitted from the laser, then the light is passed through the collimating lens, then the light is directed by the MEMS micro mirror towards the entity, then the light is passed through the second lens before the light reaches the entity.

The MEMS micro minor may be configured to oscillate about a single oscillation axis.

The projector may further comprise a diffractive optical element, and the step of using a projector to project light towards the entity to project an image which is composed of pixels onto the entity may comprise, reflecting the light using a diffractive optical element.

The diffractive optical element may be integral to a reflective of the MEMS micro minor and wherein the step of using a projector to project light towards the entity to project an image which is composed of pixels onto the entity may comprise, emitting light from the laser; passing the light through a first collimating lens which is configured to collimate light in two orthogonal axes; and directing the collimated light towards the entity using a diffractive optical element which is integral to a reflective of the MEMS micro mirror.

The projector may further comprise a speckle-reducing-optical-element which comprises a reflective layer and a semi-transparent-semi-reflective layer, and wherein the step of using a projector to project light towards the entity to project an image which is composed of pixels onto the entity may comprise, reflecting a first portion of the light, using the semi-transparent-semi-reflective layer, towards the entity; transmitting a second portion of the light though the semi-transparent-semi-reflective layer; and reflecting the second portion of the light using the reflective layer, towards the entity, so as to reduce speckle in the projected image.

The speckle-reducing-optical-element may comprises at least one of, a micro-lens array comprising a reflective layer and a beam-splitting layer; a diffractive optical element comprising a reflective layer and a beam-splitting layer; a diffractive grating comprising a reflective layer and a beam-splitting layer.

Preferably the beam-splitting layer comprises a semi-transparent-semi-reflective material.

According to a further aspect of the present invention there is provided a device for detecting the positioning of an entity, comprising, a projector, which comprises a laser and a MEMS micro minor arranged to receive light from the laser and which can oscillate about at least one oscillation axis, to project light towards the entity to project an image which is composed of pixels onto the entity, wherein the image has a first density of pixels; a controller which is configured to adjust the device so as to change the density of pixels in the projected image; a sensor which is configured to sense at least a portion of the light of the projected image which is reflected from the entity and configured to use the sensed portion of the light to determine the position of the entity.

The entity may be a human body. The entity may be a body part. The entity may be the pupil of an eye of a person.

The sensor may be further configured to determine the direction in which the person is looking from the determined position of the pupil.

The sensor may be further configured to determine movement of the entity using a plurality of determined positions of the entity.

The controller may be further configured to, identify an area of interest within the image; and to increase the density of pixels in the area of interest only.

The area of interest may be an area of the image which is projected onto an eye of the person.

The area of interest may be an area of the image which is projected onto a predefined body part of the person.

The controller may be configured to decrease the density of pixels outside of the area of interest.

The pixels may be configured to be spot shaped or line shaped.

The controller may be configured to change the laser modulation speed so as to change the density of pixels in the projected image.

The controller may be configured to change the speed at which the MEMS micro minor oscillates about its at least one oscillation axis and/or change the amplitude of oscillation of the MEMS micro mirror about its at least one oscillation axis to change the density of pixels in the projected image.

The projector may be configured to project an image which has a predefined pattern of pixels, and the sensor may comprise a plurality of light detectors which are arranged in a pattern equal to the predefined pattern of the pixels of the projected image.

The predefined pattern may be at least one of a rectangular pattern a diagonal pattern, an Archimedean spiral, a constant spaced-points spiral, and/or a star pattern.

The projector may further comprise a first lens which is configured to collimate light along a first axis, and a second lens which is configured to focus light along a second axis, wherein the first and second axes are each orthogonal to one another, and wherein the first and second lenses are located in an optical path between the laser and the MEMS micro mirror.

The first and/or second lens may be one of, a semi-cylindrical lens; a cylindrical lens; an planoconvex semi-cylindrical lens; a biconvex cylindrical lens, concavoconvex cylindrical lens as, a planoconcave semi-cylindrical lens, a biconcave cylindrical lens, or a convexoconcave cylindrical lens.

The first lens may be configured to receive light form the laser and the second lens may be configured to receive light which has passed through the first lens, and wherein the MEMS micro minor may be configured to receive light from the second lens.

The MEMS micro minor may be located at distance from the second lens equal to twice the focal length of the second lens.

The laser may be located at distance from the second lens which is equal to the distance between the MEMS micro minor and the second lens.

The projector may further comprise a collimating lens which is configured to collimate light along a first and second axis, wherein the first and second axes are orthogonal, and a second lens which is configured to focus light along a third axis. Preferably the third axes is orthogonal to each of the first and second axes. The collimating lens may be configured to collimate the light beam in a first and second axis, wherein the first and second axes are orthogonal; and the second lens may be configured to focus or diverge light along a third axis which is orthogonal to the first and second axes. It will be understood that the second lens may be configured to focus or diverge the light in any axis, and preferably is configured to focus or diverge light in an axis which is parallel to the axis of oscillation of the MEMS mirror. In other words preferably the third axis is parallel to the axis of oscillation of the MEMS minor.

The second lens may be one of, a semi-cylindrical lens; a cylindrical lens; an planoconvex semi-cylindrical lens; a biconvex cylindrical lens, concavoconvex cylindrical lens as, a planoconcave semi-cylindrical lens, a biconcave cylindrical lens, or a convexoconcave cylindrical lens

The collimating lens may be configured to receive light from the laser, and the MEMS micro minor may be configured to receive light from the collimating lens, and the second lens may be configured to receive light which is reflected by the MEMS micro mirror.

The laser may be located at a distance from the collimating lens, which is equal to the focal length of the collimating lens.

The projector may further comprise a diffractive optical element.

The diffractive optical element may be integral to a reflective surface of the MEMS micro minor.

The MEMS micro minor may comprise a collimating lens which is located in an optical path between the laser and the diffractive optical element.

The projector may further comprise a speckle-reducing-optical-element which comprises a reflective layer and a beam-splitting layer.

The speckle-reducing-optical-element may be arranged to receive light which is reflected from the MEMS micro mirror and to direct light towards the entity.

The speckle-reducing-optical-element may comprise at least one of; a micro-lens array comprising a reflective layer and a beam-splitting layer; a diffractive optical element comprising a reflective layer and a beam-splitting layer, diffractive grating comprising a reflective layer and a beam-splitting layer.

The beam splitting layer preferably comprises a semi-transparent-semi-reflective material.

The MEMS micro minor may be configured to oscillate about a single oscillation axis only.

The MEMS micro minor may be configured to oscillate about two orthogonal oscillation axis.

According to a further aspect of the present invention there is provided a method of measuring distance, comprising the steps of, (a) using a projector to project light towards an entity to project an image which composed of pixels onto the entity, wherein the image has a first density of pixels; (b) changing the density of pixels in the projected image; (c) sensing at least a portion of light of the projected image which is reflected from the entity; and (d) using the sensed portion of the light to determine the distance of the entity from the projector.

The method of measuring distance may further comprise one or more of the steps mentioned above for the method of determining position.

According to a further aspect of the present invention there is provided a device for measuring distance, comprising, a projector, which comprises a laser and a MEMS micro mirror arranged to receive light from the laser and which can oscillate about at least one oscillation axis, to project light towards the entity to project an image which is composed of pixels onto the entity, wherein the image has a first density of pixels; a controller which is configured to change the density of pixels in the projected image; a sensor which is configured to sense at least a portion of the light of the projected image which is reflected from the entity and configured to use the sensed portion of the light to determine the distance of the entity away from the projector.

A device for measuring distance may further comprise one or more of the features mentioned in the device for determining position mentioned above.

### BRIEF DESCRIPTION OF THE DRAWINGS

The invention will be better understood with the aid of the description of an embodiment given by way of example and illustrated by the figures, in which:

FIG. 1a-c provide perspective views of a device according to a first embodiment of the present invention in use; FIG. 1d provides a magnified view of the part of FIG. 1c which contains the area of interest;

FIGS. 2a-c illustrate examples of patterns of pixels in an image projected by the projector of the device shown in FIGS. 1a-c;

FIG. 3a illustrates the pixels of an image when the controller has adjusted the oscillation speed of the MEMS micro minor; FIG. 3b illustrates the pixels of an image when the controller 9 has adjusted the amplitude of oscillation of the MEMS micro minor; FIG. 3c illustrates the pixels of an image when the controller 9 has adjusted the laser modulation speed;

FIG. 4 illustrates an alternative configuration for the projector in device shown in FIG. 1a-c;

FIG. 5 illustrates an alternative configuration for the projector in device shown in FIG. 1a-c;

FIGS. 6a-h illustrate alternative lens which can be used in the projectors illustrated in FIGS. 4 and 5;

FIG. 7a illustrates an alternative configuration for the projector in device shown in FIG. 1a-c;

FIG. 7b illustrates a diffractive optical element used in the projection shown in FIG. 7a;

FIG. 8 illustrates the projector of FIG. 4 further comprising a speckle-reducing-optical-element;

FIGS. 9a and 9c-f shows a speckle-reducing-optical-element which can be used in a device according to any of embodiments of the present invention;

FIG. 9b shows alternative speckle-reducing-optical-element, in use, which can be used in a device according to any of embodiments of the present invention;

FIG. 9g shows an alternative speckle-reducing-optical-element which can be used in a device according to any of embodiments of the present invention.

### DETAILED DESCRIPTION OF POSSIBLE EMBODIMENTS OF THE INVENTION

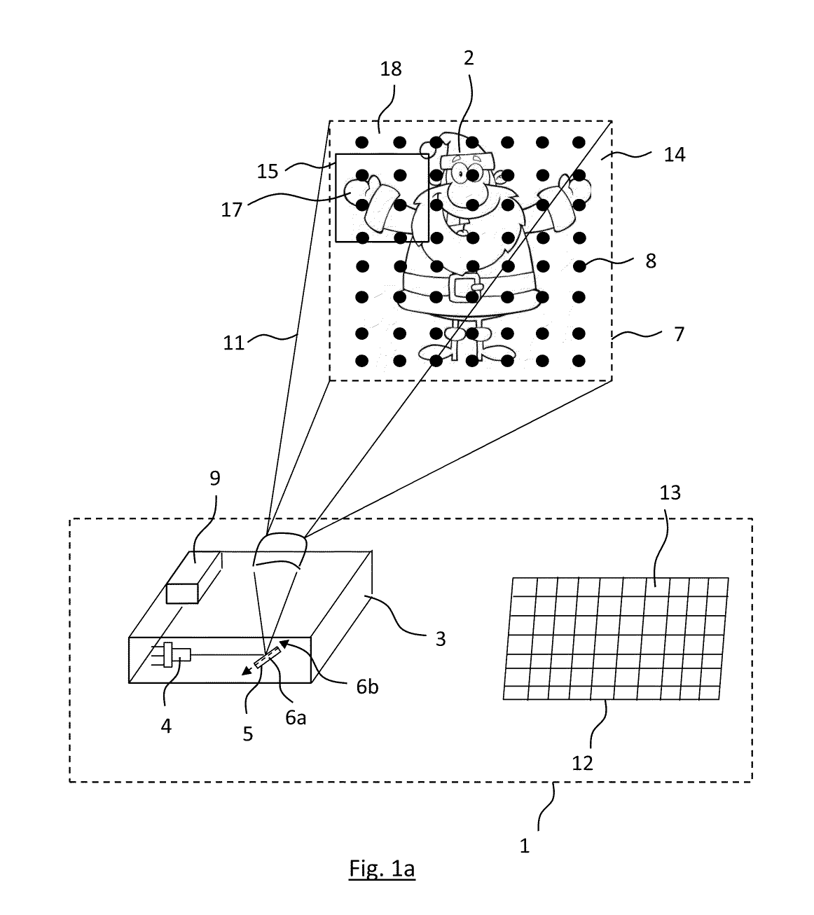

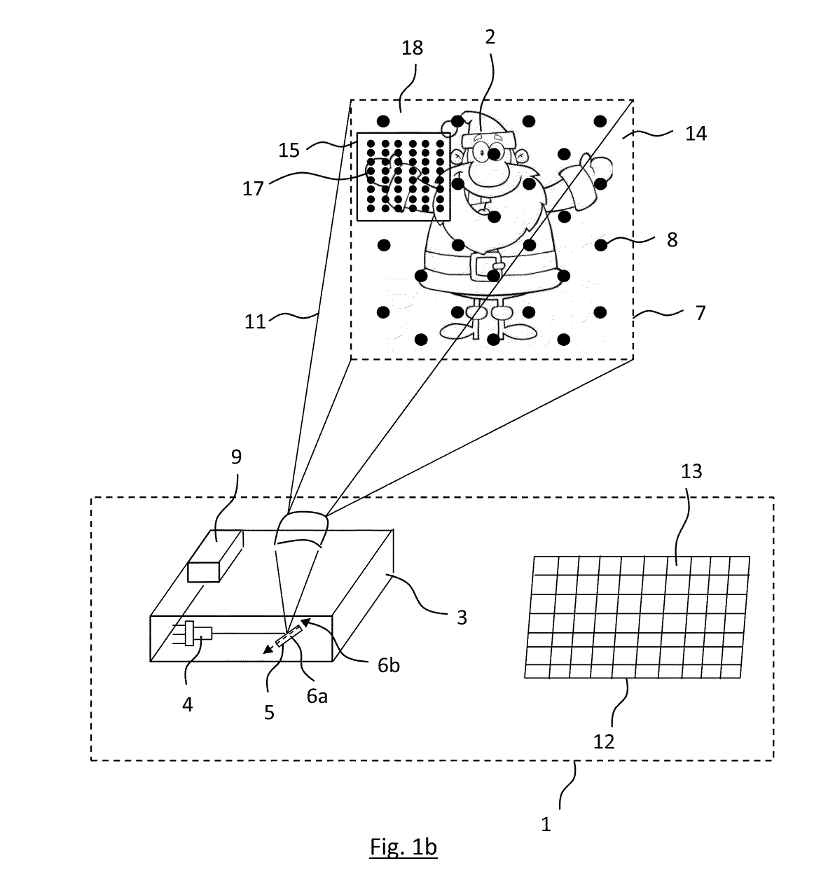

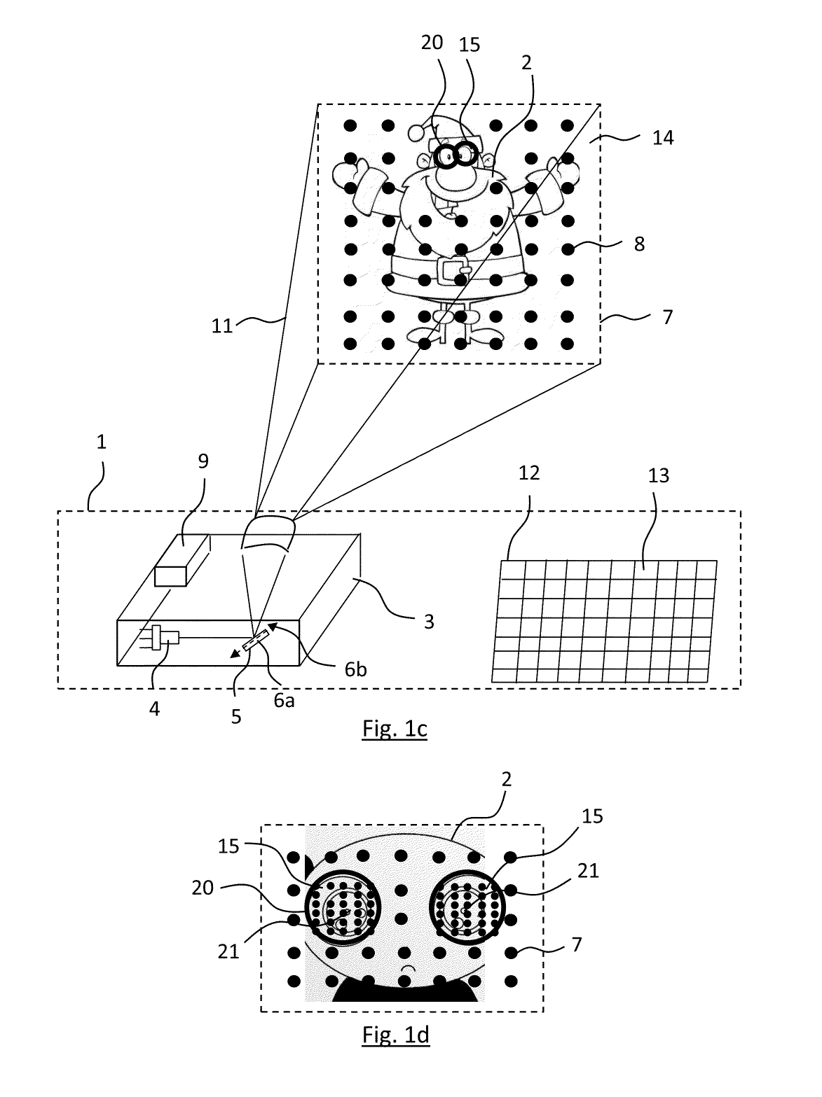

FIG. 1a,1b provide a perspective view of a device 1 for detecting the positioning of an entity 2, or the position or part of that entity 2, according to a first embodiment of the present invention.

The device 1 comprises, a projector 3, which comprises a laser 4 and a MEMS micro minor 5 arranged to receive light from the laser 4. It will be understood that any suitable light source may be used in place of a laser 4; preferably the laser 4 may be a VCSEL or laser diode or Resonant Cavity-LED or SLED. The light which is emitted from the laser 4 may be of a visible wavelength or may be Infra-Red light; in this example the laser 4 is configured to emit light which has a visible wavelength.

The MEMS micro minor 5 is configured such that it can oscillate about at least one oscillation axis 6a,b, to deflect light 11 towards the entity 2 so as to project an image 7, which is composed of pixels 8, onto the entity 2. In this example the MEMS micro minor 5 is configured such that it can oscillate about two orthogonal oscillation axis 6a,b; however it will be understood that the MEMS micro minor 5 could alternatively be configured such that it can oscillate about a single oscillation axis.

The pixels 8 are shown to be spot-shaped, and are shown to each have the same size; however it will be understood that the pixels 8 may have any suitable shape, for example elliptical, square and/or rectangular, and the pixels 8 of an image 7 may have different sizes across that image 7. The modulation speed of the laser 4, and the oscillation speed and oscillation amplitude of the MEMS micro minor 5 about its oscillation axes 6a,b, is such that there is a gap of between 0.1-1 cm between each successive pixel 8 of the image 7; and because in between the projection of two consecutive pixels the laser is turned off, no light is outputted in between two spots and this ensures that there is a high contrast between the areas of the entity 2 on which a pixel 8 is projected and the areas of the entity 2 where no pixels 8 are projected.

The device 1 further comprises a controller 9 which is configured to adjust the device 1 so as to change the density of pixels in the projected image 7. It will be understood that the controller 9 may be configured to adjust the device 1 so as to increase and/or decrease the density of pixels 8 in one or more parts, or the whole, of the projected image 7.

Additionally the device 1 comprises a sensor 12 which is configured to sense at least a portion of the light of the projected image 7 which is reflected from the entity 2. The sensor 12 is configured to use the sensed portion of the light to determine the position of the entity 2 or to determine the position of a part of the entity 2. In this example the sensor 12 comprises a matrix of discrete light detectors 13. Advantageously the higher contrast between the areas of the entity 2 on which a pixel 8 is projected and the areas of the entity 2 where no pixels 8 are projected, which is due to the gap of between 0.01-1 cm between each successive pixels 8 of the image 7, will reduce the sensitivity requirement of a sensor 12 since the signal to noise ration of the light reflected from the entity 2 will be increased.

In particular the device 1 is operable to determine the position of the entity 2, or part of the entity 2, within a predefined area 14 (i.e. field of view). The predefined area 14 is the area over which the image 7 is projected. In a preferable embodiment the projected light 11 comprises discrete light beams each of which defines a respective pixel 8 of the projected image 7. Those discrete light beams which are projected onto the entity 2 will be reflected by the entity 2; and those reflected light beams are then received at the sensor 12. The position of the entity 2, or the part of the entity 2, within the predefined area 14 is then determined based on which of the discrete light detectors 13 received the reflected discrete light beams. For example, if the discrete light detectors 13 present in the centre of the sensor 12 receive more the reflected light beams than those light detectors 13 which are present at the fringes of the sensor 12, this will indicate that the entity 2, or the part of the entity 2, is in the centre of the predefined area 14.

The sensor 12 is further configured to determine movement of the entity 2, or part of the entity 2, from a series of successive determined positions. For example at a first time instance, the sensor 12 receives discrete light beams which are reflected by the entity 2, or part of the entity 2, and determines a first position of the entity based on which of the discrete light detectors 13 receive the most reflected discrete light beams; and at a second time instance, the sensor 12 receives discrete light beams which are reflected by the entity 2, or part of the entity 2, and determines a second position of the entity based on which of the discrete light detectors 13 receive the most reflected discrete light beams; movement of the entity 2, or part of the entity 2, may be determined by subtracting the first position from the second position.

The sensor 12 may be further configured to recognise predefined movements (e.g. gestures which the entity 2 makes) and to initiate a predefined action in response to recognising a predefined movement (e.g. to initiate a predefined movement of a character in video game in response to recognising a predefined movement).

In another embodiment the sensor 12 may additionally, or alternatively, be configured to determine the distance between the entity 2 and the projector 3 using light which it receives (i.e. the light which has been projected by the projector 3 and reflected by the entity 2 to the sensor 12). The sensor 12 may be configured to determined distance using time of flight of the light which is emitted from the projector, reflected from the entity and received at the sensor 12, or using phase changes in the light which is emitted from the projector, reflected from the entity and received at the sensor 12, as is well known in the art. The manner in which the sensor 12 may be configured to determined distance using time of flight of the light or phase changes in the light is known in the art. Many other techniques for determining distance using reflected light are known in the art, and any of the sensor 12 may be configured to determine distance using any of these techniques.

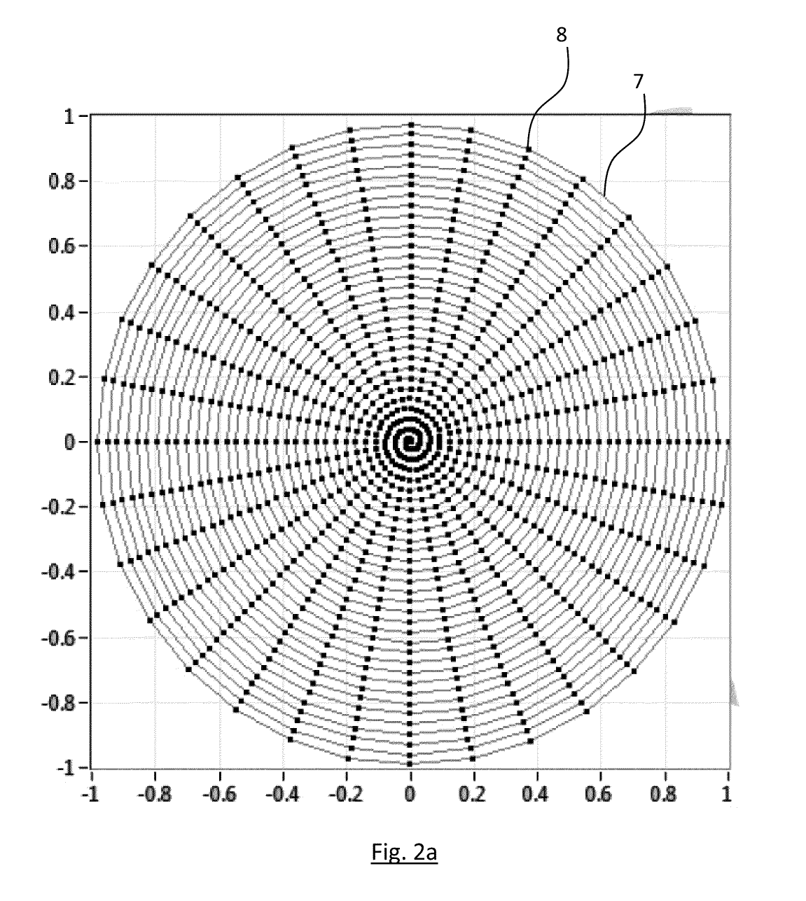

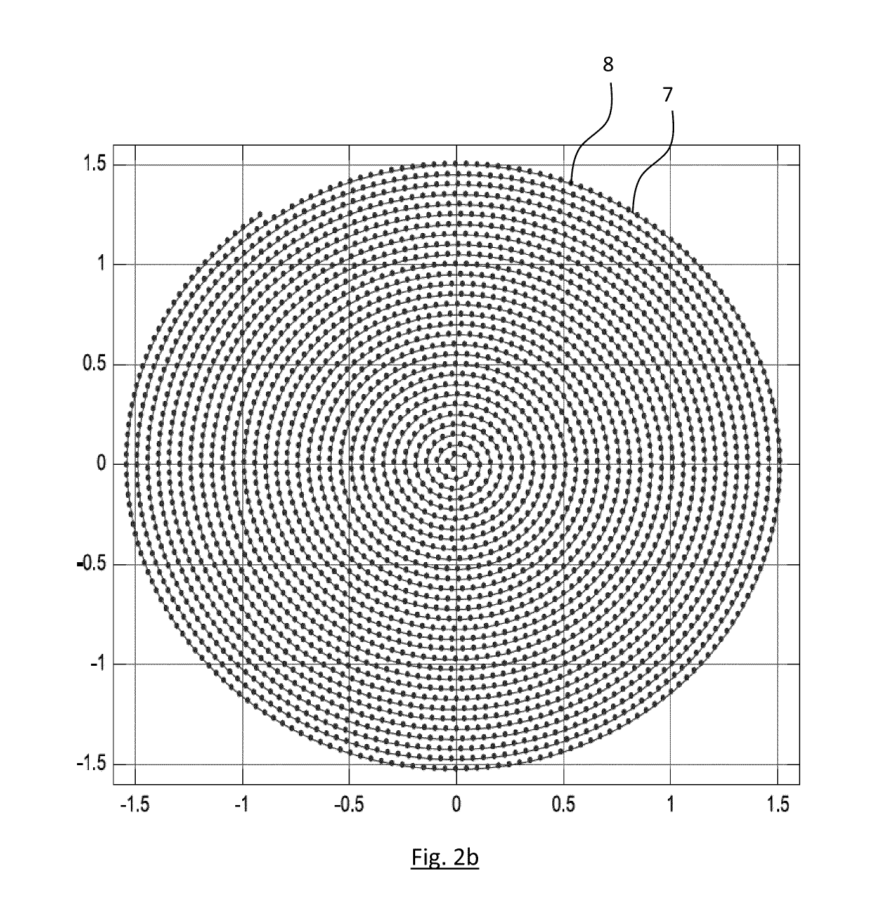

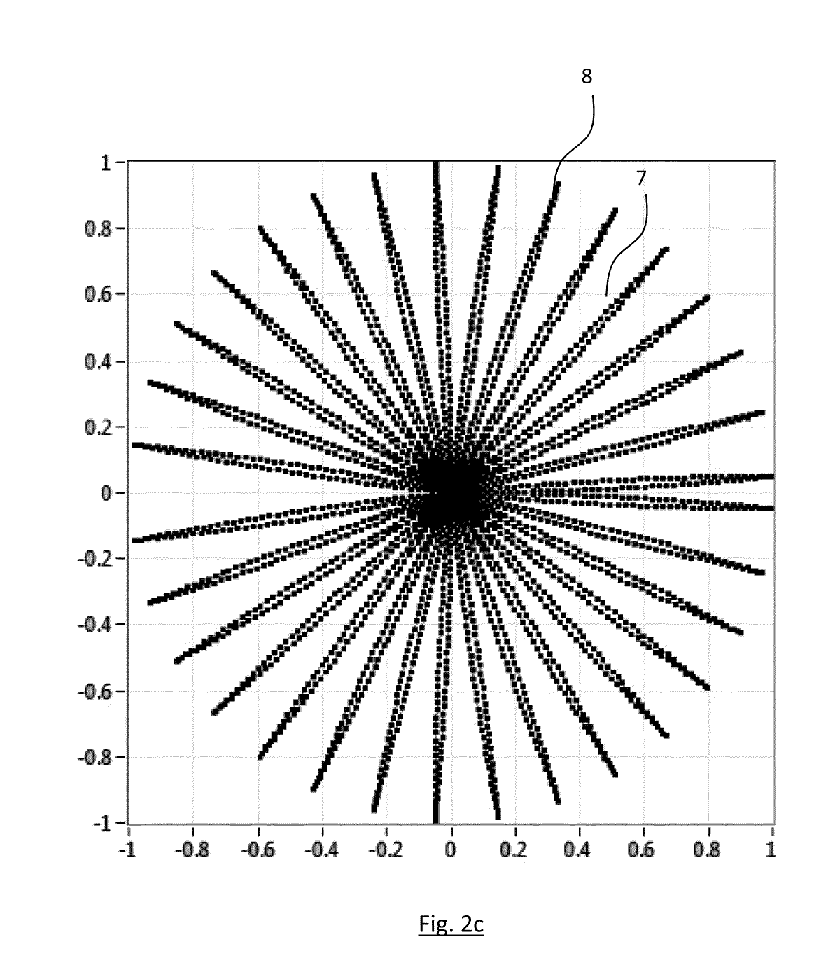

The projector 3 of device 1 is configured to project an image 7 which has a predefined pattern of pixels 8. The sensor 12 is configured such that its discrete light detectors 13 are arranged in a pattern equal to the predefined pattern of the pixels 8. Advantageously this will provide for more efficient, simplified, and more accurate determination of the position of the entity 2, or part of the entity 2. In the example illustrated in FIGS. 1a,1b, the projector 3 is configured to project an image 7 which has pixels 8 in a rectangular pattern, with the pixels 8 aligned along the horizontal and vertical; correspondingly the discrete light detectors 13 of the sensor 12 are arranged in a rectantular pattern, with the discrete light detectors 13 aligned along the horizontal and vertical. It will be understood that the projector 3 of device 1 could be configured to project an image 7 which has any predefined pattern of pixels 8, such as an image 7 which has pixels 8 arranged in diagonal lines for example. Further examples of other pixel patterns are shown in FIGS. 2a-c; FIG. 2a shows the pixels 8 of the image 7 in an Archimedean spiral FIG. 2b shows the pixels 8 of the image 7 in a constant spaced-points spiral FIG. 2c shows the pixels 8 of the image 7 in a star pattern. Correspondingly it will be understood that the discrete light detectors 13 of the sensor 12 may be arranged in diagonal lines, an Archimedean spiral, constant spaced-points spiral, or a star pattern.

As mentioned the device 1 comprises a controller 9 which is configured to adjust the device 1 so as to change the density of pixels in the projected image 7. The controller 9 is further configured to identify an area of interest 15 within the image 7. Preferably the controller 9 will comprise a sensor, that senses the distances between the projector and the surface of the entity on which the image 7 is projected; the controller 9 will also preferably comprise a processing unit that is configured to receive distance information from the sensor, and to process that distance information to identify the area of interest 15 within the image 7. In the example shown in FIGS. 1a,1b the controller 9 is configured to adjust the device 1 so as to increase the density of pixels 8 in the area of interest 15 only. FIG. 1b illustrates the image 7 after the controller 9 has adjusted the device 1 to increase the density of pixels in the area of interest 15 only. Thus, the density of pixels 8 in the area of interest 15 of image 7 projected in FIG. 1b is higher than the density of pixels in the area of interest 15 of image 7 projected in FIG. 1a.

In the example shown in FIGS. 1a,1b the entity 2 is a person and the area of interest 15 is an area of the image 7 which is projected onto a predefined body part of that person, which in this case is the hand 17 of the person. It will be understood that the area of interest 15 could be any area in the image 7; and the area of interest 15 usually dependent on the application of the present invention. The embodiment shown in FIGS. 1a,1b is typically used in a video gaming application, whereby the characters of the video game are controlled by hand movements made by the person; accordingly the area of interest 15 is the area of the image 7 which is projected onto the hand 17 of the person.

Advantageously, by increasing the density of pixels within the area of interest 15 enables a more accurate determination of the hand 17 position. Accordingly a more accurate determination of the movement of the hand 17 can be achieved.

In another embodiment in which the sensor 12 is additionally, or alternatively, configured to determine distance, a more accurate measurement of the distance between the hand 17 and the projector 3 can be achieved due to the increase in pixel density in the area of interest 15. This is because a larger number of light beams are reflected by the hand and thus more reflected light is received by the sensor to be used in the determination of distance.

In the example shown in FIGS. 1a, 1b, the controller 9 is further configured to adjust the device 1 so as to decrease the density of pixels 8 in the area 18 of the image 7 which are outside of the area of interest 15. FIG. 1b illustrates the image 7 after the controller 9 has adjusted the device 1 to decrease the density of pixels in the area 18 of the image 7 which are outside of the area of interest 15.

Advantageously, decreasing the density of pixels 8 in the area 18 of the image 7 which are outside of the area of interest 15 reduces the amount of computation performed by the sensor 12. Also it provides for a more efficient device 1.

In another embodiment in which the sensor 12 is additionally, or alternatively, configured to determine distance, more efficient operation is achieved as the senor uses less processing power in determining distances using light reflected from area 18 outside of the area of interest 15.

The manner in which the controller 9 adjusts the device 1 so as to increase the density of pixels 8 in the area of interest 15 and/or to decrease the density of pixels 8 in the area 18 of the image 7 which are outside of the area of interest 15, can be achieved a plurality of ways. The controller 9 may, change the modulation speed of the laser 4; change the oscillation speed of the MEMS mirror 5 about its one or more of its oscillation axes 6a,b; change the amplitude of oscillation of the MEMS minor 5 about one or more of its oscillation axes 6a,b; or a combination of any two of these ways or; a combination of all three ways. It will be understood that this is also the case for devices 1 which have projectors 3 which have one or more MEMS micro minors 5 which oscillate about a single oscillation axis.

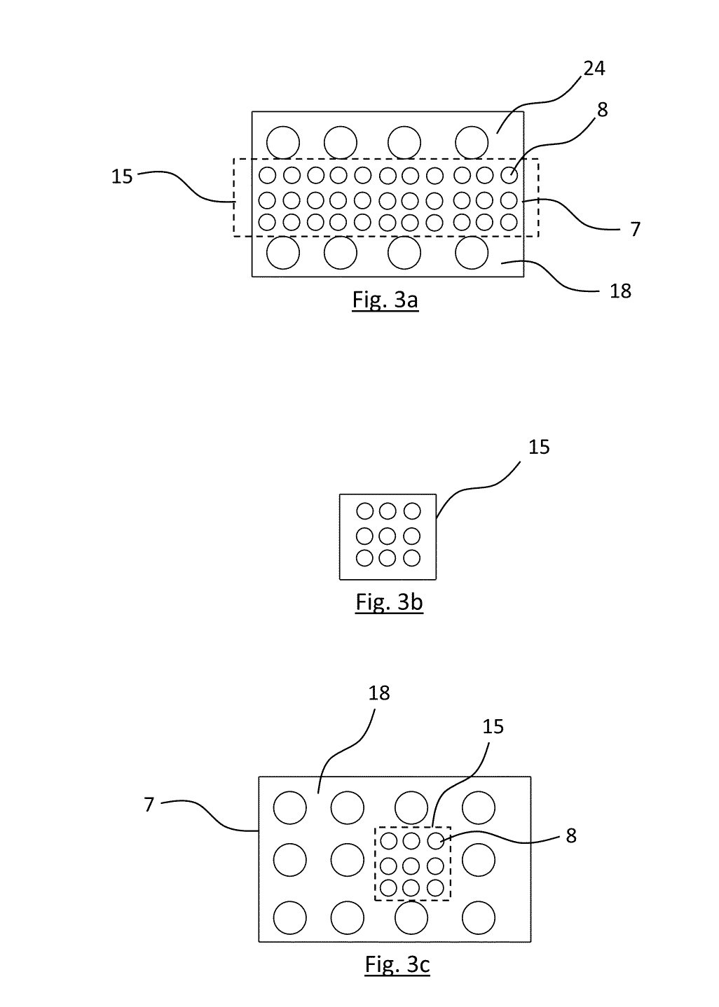

For example, the controller 9 may be configured to change the laser modulation speed/time of the laser 4 so as to adjust the device 1. The laser modulation speed is the speed at which the laser 4 is the speed at which the laser outputs consecutive light beams, each of light beams defining an independent pixel 8 of the image 7. By increasing the modulation speed when the MEMS mirror 5 is orientated to direct light to the area of interest, will increase the number of pixels 8 which are projected into the area of interest 15 Likewise by decreasing the modulation speed when the MEMS mirror 5 is orientated to direct light to the area 18 outside of the area of interest 15 will decrease the number of pixels 8 which are projected into the area 18 outside of the area of interest 15. FIG. 3c illustrates the pixels 8 of an image 7 when the controller 9 has increased the laser modulation speed when the MEMS micro mirror 5 is orientated between 0 to −30° of its oscillation about both its axes of oscillation 6a,b, to increase the number of pixels in the area of interest 15. The controller 9 has also decreased the laser modulation speed when the MEMS micro minor 5 is orientated between +45° and 0° and −30° and −45° about both its axes of oscillation 6a,b, to decrease the number of pixels outside the area of interest.

Additionally, or alternatively, the controller 9 may be configured to change the speed at which the MEMS micro minor 5 oscillates about one or more of its oscillation axes 6a,b (or about its single oscillation axis, if the MEMS micro minor 5 is configured to oscillate about a single axis) and/or to change the amplitude of oscillation of the MEMS micro minor 5. For example, if the MEMS micro mirror 5 oscillates between +−45° about its oscillation axis 6a (the oscillation axis about which the MEMS micro mirror 5 to scan light along the horizontal), and the area of interest 15 is an area at the centre of the image 7, the controller 9 may adjust the speed of oscillation of the MEMS micro minor 5 so that the MEMS micro minor 5 oscillates faster between −80° and −90°, and between +40° and +45°, and oscillates slower between −40° and +40°. This will ensure that the MEMS micro minor 5 is oscillating faster when it is projecting pixels 8 which are near the edge 24 (i.e. the area 18 outside of the area of interest) of the image 7, and MEMS micro mirror 5 is oscillating slower when it is projecting pixels which are near the centre of the image (i.e. in the area of interest 15); as illustrated in FIG. 3a, this will ensure that there is a higher density of pixels 8 provided near the area of interest 15 which is at the centre of the image 7 and a lower density of pixels 8 in the area 18 outside of the area of interest i.e. near the edge of the image 7. Similarly, the oscillation speed of the MEMS micro minor 5, during a portion of its oscillation amplitude about one or more of its oscillation axes 6a,b, may be adjusted so as to obtain an increase in pixel 8 density in any other part of the image 7 which is the area of interest 15, and optionally to also obtain a decrease in pixel density in the other parts of the image 7 outside of the area of interest 15.

Additionally, or alternatively the controller 9 may be configured to adjust amplitude of oscillation of the MEMS micro mirror 5 to achieve an increase in the density of pixels 8 in the area of interest 15. For example, the controller 9 may be configured to decrease the amplitude of oscillation of the MEMS micro mirror 5 so that all of the pixels 8 of the image 7 are projected to within a smaller area of the image 7 which is the area of interest 15, as is illustrated in FIG. 3b.

Thus, by changing one or more of the laser modulation speed, speed of oscillation of the MEMS micro mirror 5, and/or the amplitude of oscillation of the MEMS micro mirror 5, the controller 9 adjusts the device 1 so as to increase the density of pixels in the area of interest 15 and/or to decrease the density of pixels 8 in the area 18 of the image 7 which is outside of the area of interest 15.

Referring to FIGS. 1c and d; FIG. 1c shows the device 1 in use in an application in which the area of interest 15 is an area of the image 7 which is projected onto the eyes 20 of the person 2. A magnified view of the part of FIG. 1c, which contains the area of interest 15 with an increased density of pixels 8, is illustrated in FIG. 1d.

The device 1 can be used to determine the position of the pupil 21 of an eye 20. The sensor 12 may be further configured to determine the direction in which the person 2 is looking based on the determined position of the pupil 21 of an eye. An example of an application in which the area of interest 15 is the area of the image 7 which is projected onto the eyes 20 of the person 2, is use in internet marketing to determine which part of the screen a person is looking at so as to determine if a person is viewing an advert which is displayed on part the screen.

The density of pixels 8 in the area of interest 15 is increased in the manner previously described with respect to FIGS. 1a, 1b, 3a-c.

As mentioned, the projected light 11 comprises discrete light beams each of which defines a respective pixel 8 of the projected image 7. Those discrete light beams which are projected onto the pupil 21 of an eye 20 will be absorbed into the eye 20, while those discrete light beams which are projected onto the areas of the eye 20 outside of the pupil 21 are reflected by the eye 20. Those reflected light beams are received at the discrete light detectors 13 of the sensor 12. The position of the pupil 21 of an eye 20 is then determined based on which of the discrete light detectors 13 received the reflected discrete light beams and/or based on which of the discrete light detectors 13 do not receive reflected discrete light beams. For example, if the discrete light detectors 13 in the centre of the sensor 12 receive no reflected light beams this will indicate that the pupils 21 are located in the centre of the eyes 20; the light detectors 13 in the centre of the sensor 12 will receive no reflected light beams all the discrete light beams which would otherwise be reflected the discrete light detectors 13 at centre of the sensor 12 have been absorbed into the pupils 21 of the eyes 20. Based on the determined position of the pupil 21 the sensor 12 can determined the direction in which the person 2 is looking.

The sensor 12 may be further configured to determine movement of the pupils 21 from a series of successive determined positions and thus can determine changes in the direction in which a person 2 is looking. For example, at a first time instance, the sensor 12 receives the discrete light beams which are reflected by the eyes 20 of the person 2 and determines a first position of the pupils 21 based on which of the discrete light detectors 13 receive the reflected discrete light beams and/or which do not receive the reflected discrete light beams; and at a second time instance, the sensor 12 receives discrete light beams which are reflected by the eyes 20 of the person 2 and determines a second position of the pupils 21 based on which of the discrete light detectors 13 receive the reflected discrete light beams and/or which do not receive the reflected discrete light beams; movement of the pupils 21 may be determined by subtracting the first position from the second position, thus changes in the direction in which the person 2 is looking can be determined by subtracting the first position from the second position.

In a further application the device 1 there may be two or more areas of interest 15. For example, in a further application the device 1 may, consecutively, operate as illustrated in FIGS. 1a,b for a first predefined period and then operate as illustrated in FIGS. 1c,d for a second predefined period. The device 1 may operate to continually switch between the two different modes of operation. Thus the device 1 will determine the position of the hand 17 of the person 2 during the first predefined period and then determine the position of the pupils 21 of the person 2 during the second predefined period. The sensor 12 may be synchronized with the projector 3 so that it performs the necessary steps to determine the position of the hand 17 during the first predefined period and performs the necessary steps to determine the position of the pupils 21 during the second predefined period.

In a variation of the invention the device 1 may comprise a plurality of sensors 12. Each of the plurality of sensors 12 may determine the position of a different entities or different part of the same entity; for example the device 1 may comprise a first sensor 12 which determines the position of the hand 17 of the person 2 and a second sensor 12 which determines the position of the pupils 21 of the person.

The light which defines the pixels 8 which are in the area 18 outside of the area of interest 15 will be reflected by other parts of the entity 2 and/or by objects or surfaces which are around the entity 2. The device 1 may further comprise a sensor which receives this reflected light. The sensor may further be configured to detect changes in this reflected light. The changes in this reflected light may be used to indicate changes occurring in the environment around the entity 2; for example if a person moves into the region close to the entity i.e. if a person moves into the area of the image 7 (or more specifically if the person moved into the projection cone defined by the light 11 which is projected by the projector 3).

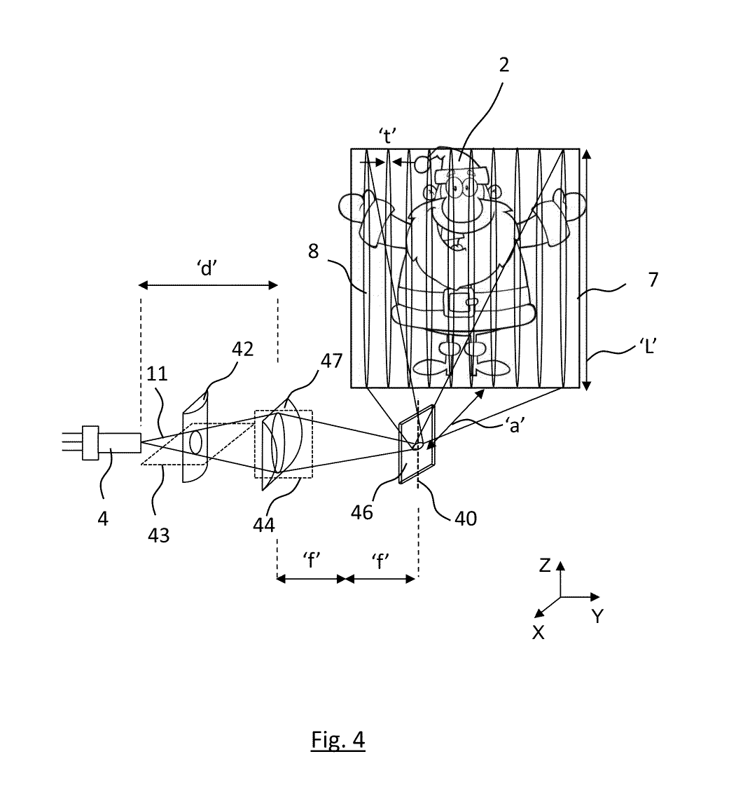

In the projector 3 of the device 1 shown in FIGS. 1a-c the MEMS micro mirror 5 is preferably configured to oscillate about at two orthogonal oscillation axes 6a,b so that the MEMS micro minor 5 can oscillate to scan light in two dimensions; this will enable the pixels 8 of the image 7 to be projected consecutively, in their two dimensional rectangular pattern, onto the entity 2. Oscillation of the MEMS micro mirror 5 about one of the axes 6a of oscillation scans the light 11 along the horizontal and oscillation of the MEMS micro mirror 5 about the other axes 6b of oscillation scans the light 11 along the vertical. It should be understood however that other configurations for the projector 3 are possible. FIGS. 4, 5 and 7 each illustrate other possible configurations for the projector 3 in device 1.

As shown in FIG. 4, the projector 3 may alternatively comprise a MEMS micro minor 46 which can oscillate about a single oscillation axis 40; a first lens in the form of a first semi-cylindrical lens 42 which is configured to collimate light, which is received from the laser 4, along a first collimation plane 43, and a second lens in the form of a second semi-cylindrical lens 47 which is configured to focus light, which it receives from the first semi-cylindrical lens 42, along a second collimation plane 44. The second collimation plane 44 is perpendicular to the first collimation plane 43. The first semi-cylindrical lens 42 is arranged to receive light 11 from the laser 4; the second semi-cylindrical lens 47 is arranged to receive light which has passed through the first semi-cylindrical lens 42; the MEMS micro mirror 46 is arranged to receive light which has passed through the second semi-cylindrical lens 47. The first semi-cylindrical lens 42 and the second semi-cylindrical lens 47 are arranged so that their collimation planes 43,44 are perpendicular to one another. It will be understood that a collimation plane of a semi-cylindrical lens is the plane in which is included the smallest radius of curvature of the lens cross section.

The MEMS micro minor 46 is positioned at distance from the second semi-cylindrical lens 47 which is equal to twice the focal length ‘f’ of the second semi-cylindrical lens 47. The MEMS micro minor 46 is further preferably orientated such that it makes an angle of 45° with the second collimation plane 44. The laser 4 is located at a distance ‘d’ from the second semi-cylindrical lens 47; the distance ‘d’ is preferably equal to the distance between the second semi-cylindrical lens 47 and the MEMS micro minor 46; thus the distance ‘d’ is preferably equal to twice the focal length ‘f’ of the second semi-cylindrical lens 47.

During use of the device 1, light 11 output from the laser 4 is received at the first semi-cylindrical lens 42 where the light 11 is collimated along a first collimation plane 43; the collimated light 11 then passes through the second semi-cylindrical lens 47 where it is focused along a second collimation plane 44 which is perpendicular to the first plane collimation 43; the MEMS micro minor 46 receives the focused light from the second semi-cylindrical lens 47 and directs the light towards the entity 2 to project a pixel 8 of the image 7 onto the entity 2. The MEMS micro minor 46 oscillates about its single axis of oscillation 40 to project the pixels 8 of the image 7 consecutively onto the entity 2. In this particular embodiment the pixels 8 of the image 7 are elongated elliptical shaped such that they appear almost as lines. The length ‘1’ of each of the elongated elliptical shaped pixel 8 will depend on the divergence of the light beam which is projected from the MEMS micro minor 46 to the entity 2 and is therefore dependent on the distance ‘Q’ between the MEMS micro mirror 46 and the entity 2. The divergence angle of the light beam which is projected from the MEMS micro mirror 46 to the entity 2 depends on the focal length of the second semi-cylindrical lens; a smallerfocal length will provide a larger divergence angle while a larger focal length will provide a smaller divergence angle. The thickness ‘t’ of each of the elongated elliptical shaped pixel 8 will depend on the modulation speed or modulation time of the laser 4.

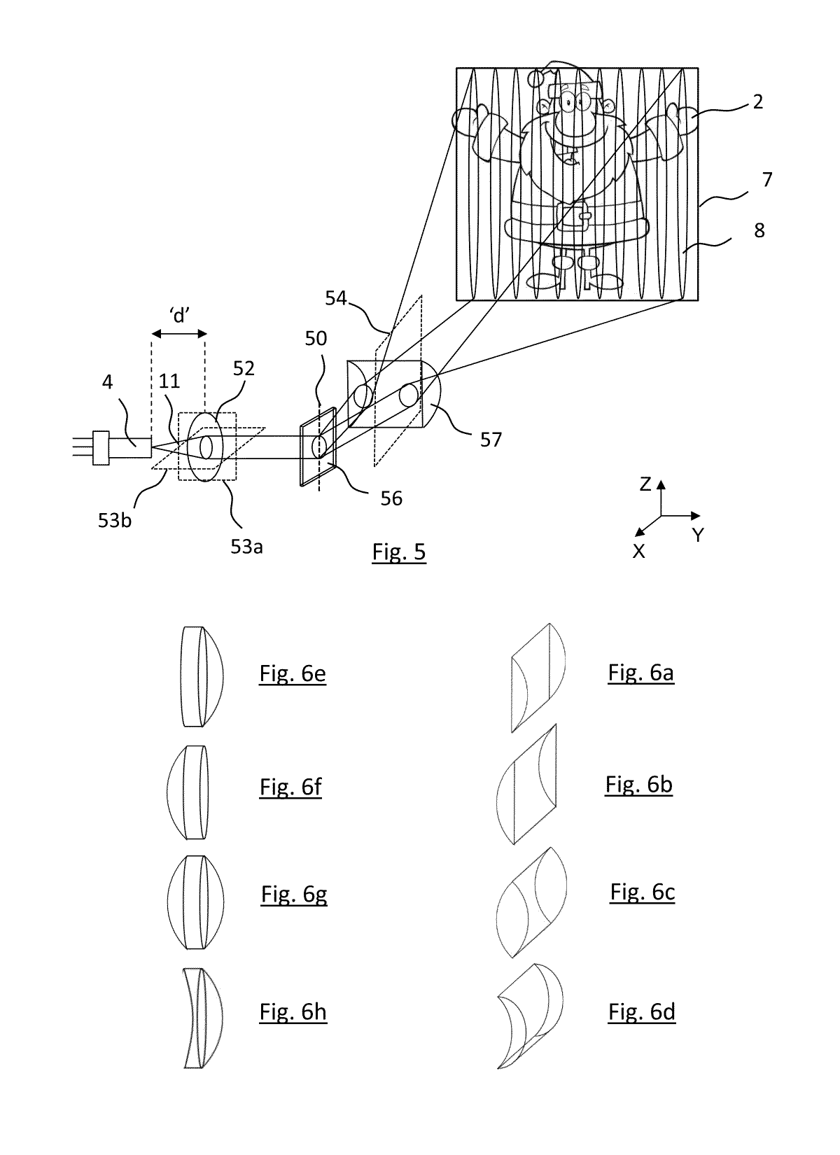

As illustrated in FIG. 5, the projector 3 of the device 1 may alternatively comprise a MEMS micro mirror 56 which can oscillate about a single oscillation axis 50; a spherical lens 52 which is configured to collimate light along a first and second orthogonal plane 53a,b, and a second lens in the form of a semi-cylindrical lens 57 which is configured to focus light along a third plane 54. The third plane 54 is perpendicular to the first and second planes 53a,b. The laser 4 is positioned at a distance ‘d’ from the spherical lens 52; most preferably the distance ‘d’ is equal to the focal length of the spherical lens 52. The spherical lens 52 is arranged to receive light 11 form the laser 4; the MEMS micro minor 56 is arranged to receive light 11 from spherical lens 52 and to direct the light 11 it receives in the direction of the entity 2; the semi-cylindrical lens 57 is arranged to receive light 11 which is reflected by the MEMS micro minor 56 and the light passes through the semi-cylindrical lens 57 before being projected onto the entity 2.

In a further variation of the projector 3, as illustrated in FIG. 5, an aspherical lens may be provided instead of the spherical lens 52.

In each of the variations of the different configurations for the projector 3 shown in FIGS. 4 and 5 is should be understood that another suitable lens type could be provided instead of any of the semi-cylindrical lenses 47,42,57. Examples of other suitable lens types are illustrated in FIG. 6a-d; these include an planoconvex semi-cylindrical lens as shown in FIGS. 6a and 6b; a biconvex cylindrical lens as shown in FIG. 6c, a concavoconvex cylindrical lens as shown in FIG. 6d, a planoconcave semi-cylindrical lens, a biconcave cylindrical lens or a convexoconcave cylindrical lens.(. It should also be understood that another suitable lens type could be provided instead of the spherical lens 52 in the projector 3 shown in FIG. 5. Examples of other suitable lens types are illustrated in FIG. 6e-h these include a planoconvex lens as shown in FIGS. 6e and 6f; a biconvex lens as shown in FIG. 6g, or concavoconvex lens as shown in FIG. 6h.

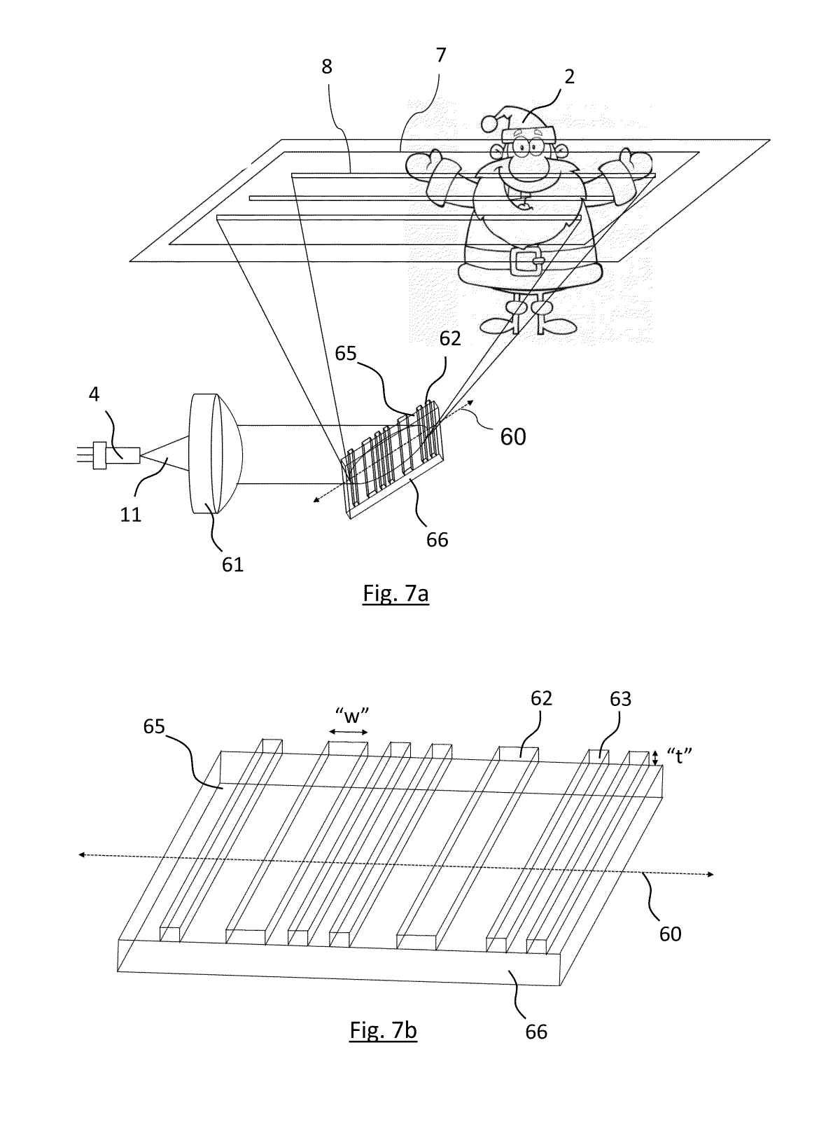

As illustrated in FIG. 7a, the projector 3 of the device 1 may alternatively comprise a MEMS micro mirror 66 which can oscillate about a single oscillation axis 60; a collimating lens 61; and a diffractive optical element 62. In this particular example the diffractive optical element 62 is integral to a reflective surface 65 of the MEMS micro mirror 66, however it should be understood that the diffractive optical element 62 may alternatively be provided as a component which is mechanically independent of the MEMS micro minor 66. The collimating lens 61 is arranged to receive light 11 from the laser 4; and the MEMS micro mirror 66 is arranged so that the diffractive optical element 62, which is integral to its reflective surface 65, receives light 11 which has passed through the collimating lens 61. If the diffractive optical element 62 is provided as a component which is mechanically independent of the MEMS micro mirror 66 then the diffractive optical element 62 may alternatively be arranged to receive light from the MEMS micro minor 66 and to direct light 11 it receives to the entity 2, or alternatively arranged to receive light from the collimating lens 61 and to direct light 11 it receives to the MEMS micro minor 66.

FIG. 7b shows a detailed view of the diffractive optical element 62 of the projector 3 shown in FIG. 7a. In this example the diffractive optical element is a reflective structure and is shaped to have a plurality of projections 63; the plurality of projections 63 have two or more different heights “t”. The projections 63 are shaped and arranged so that the diffractive optical element diffracts light it receives to shape that light so that the light can form line(s), dot(s) or any predefined shaped pixel when projected onto the entity. In this example the projections 63 are shaped as rectangular strips 63 which have a rectangular or square cross section. I a variation of this embodiment the diffractive optical element 62 may be configured to be mechanically independent of the MEMS micro mirror 66 and may be located in the optical light path before or after the MEMS micro mirror 66; in this case the diffractive optical element 62 may be configured to be transparent instead of reflective.

To form line shaped pixels line, the rectangular strips 63 of the diffractive optical element 62 should be arranged to be perpendicular to the line shaped pixels to be created. The diffraction of light from each of the strips 63 of diffractive optical element 62, follows the law of diffraction W*sin(a)=n*l, where ‘a’ is the diffraction angle, ‘1’ is the wavelength of the light, ‘n’ is an integer and ‘W’ is the width of the strip 63. The smaller ‘W’ is the larger is the diffraction angle. To obtain visible diffraction (large enough ‘a’), the size of the rectangular strips 63 shall have the width (W) of 0.1 to 1000 times the wavelength of the light which is incident on the strip 63 of the diffractive optical element 62. The width “W” of each rectangular strips 63 is preferably in the order of magnitude of 0.1 to 1000 times the wavelength of the light to have visible diffraction of light. The strips 63 may be of the same widths or of different widths. Light incident on each of the rectangular strips 63 of the diffractive optical element 62 is diffracted in a direction which is perpendicular to the longest side of the rectangular strip 63. As a result a line shaped pixel is generated from the light beams diffracted by the diffractive optical element 62.

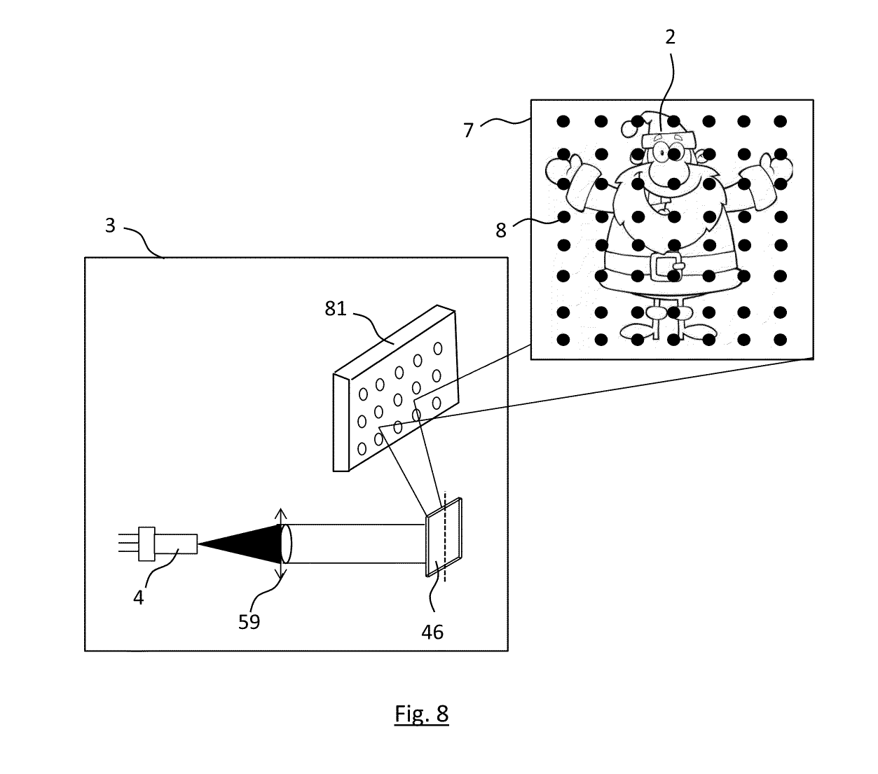

The projector 3 in any of the above-mentioned device embodiments (including embodiments shown in FIGS. 1a-c, 4, 5 and 7a-b) may further comprise a speckle-reducing-optical-element. The speckle-reducing-optical-element is preferably arranged to receive light from the MEMS micro minor and to direct the light it receives to the entity 2 so that an image 7 is projected onto the entity 2, as is illustrated in FIG. 8, which shows the projector 3 with the features of the projector 3 shown in FIG. 4 and including a speckle-reducing-optical-element 81.

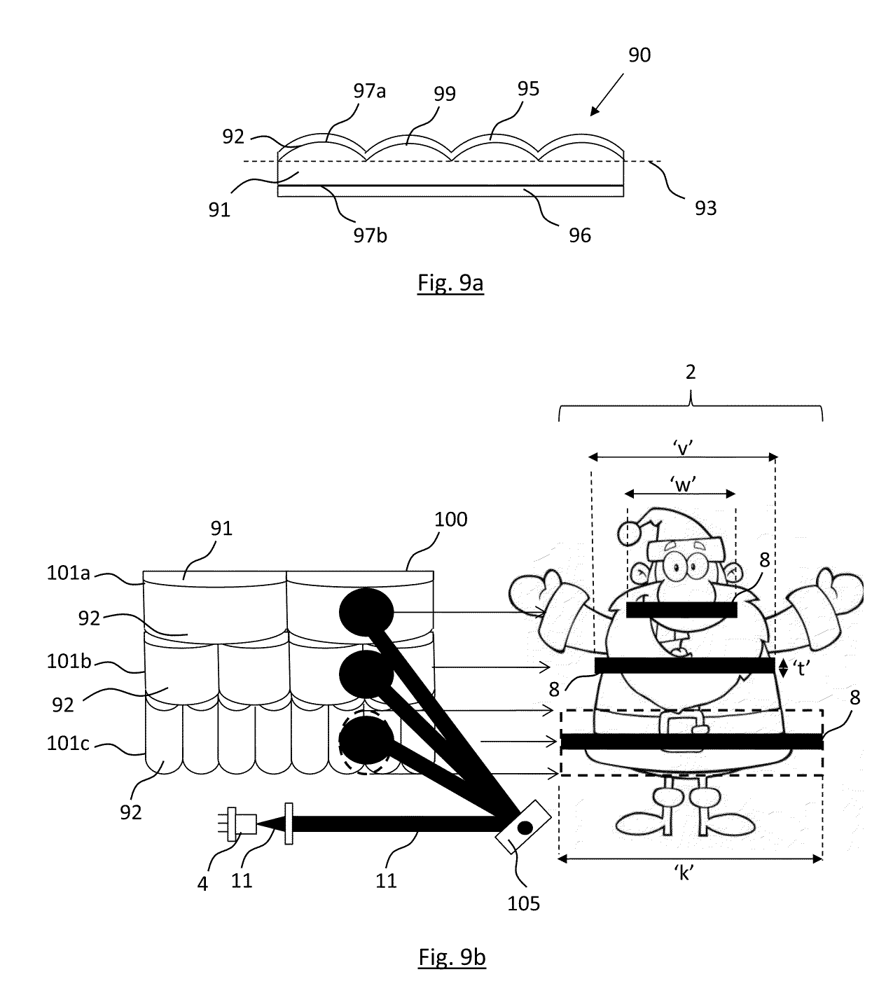

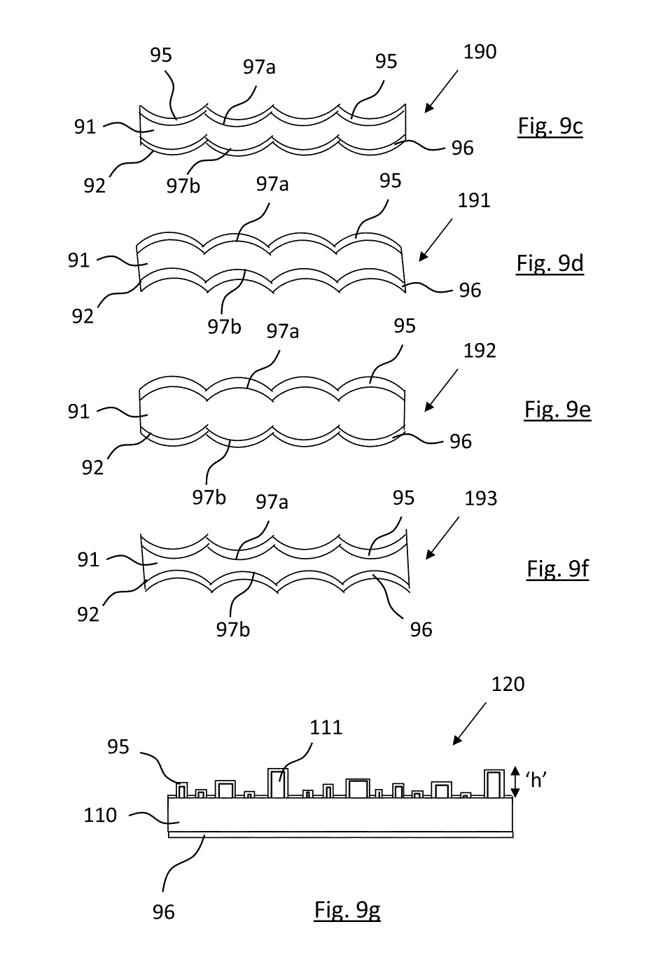

FIG. 9a provides a cross section view of a suitable speckle-reducing-optical-element 90 which can be used in the projector 3. The speckle-reducing-optical-element 90 comprises a micro-lens array 91 which comprises a plurality of micro-lens 92. The micro-lenses 92 in the micro-lens array 91 each have a convex-shaped surface 99. All of the micro-lens 92 in the micro-lens array 91 are arranged to lie on the same, single, plane 93. The speckle-reducing-optical-element 90 further comprises a beam-splitting layer 95 (i.e., a layer which can split beams); the beam-splitting layer 95 typically comprises a semi-transparent-semi-reflective material. The speckle-reducing-optical-element 90 further comprises a reflective layer 96. The beam-splitting layer 95 and reflective layer 96 are provided on opposing surfaces 97a,b of the micro-lens array 91. In this example the beam-splitting layer 95 is provided on a surface 97a of the micro-lens array 91 which is defined by the convex surfaces 99 of the micro-lenses 92, while the reflective layer 96 is provided on a flat surface 97b of the micro-lens array 91 which is opposite to the surface 97a.

In the above example of a speckle-reducing-optical-element 90 each of the micro-lens 92 in the micro-lens array 91 are equal dimensions. In a variation of the embodiment the micro-lens 92 in the micro-lens array 91 of the speckle-reducing-optical-element 90 may have different dimensions, as illustrated in FIG. 9b.

FIG. 9b shows a speckle-reducing-optical-element 100 which comprises a micro-lens array 91 which has micro-lens 92 of different dimensions. The micro-lenses 92 which are in a first row 101a of the micro-lens array 91, are configured to have larger dimension than the micro-lens 91 in a second row 101b of the micro-lens array 91; and the micro-lens 92 which are in the second row 101b of the micro-lens array 91 are in turn configured to have larger dimension than the micro-lens 91 in the last row 101c of the micro lens array 91. As was the case for the speckle-reducing-optical-element 90 shown in FIG. 9a, the speckle-reducing-optical-element 100 further comprises a beam-splitting layer which is provided on a surface of the micro-lens array 91 is defined by the convex surfaces of the micro-lenses 92, while the reflective layer is provided on an opposite, flat, surface of the micro-lens array 91. It will be understood that the surface of the micro-lens array 91 on which the reflective layer is provided may alternatively be contoured, for example that surface may comprise a micro-lens array which has concave or convex lenses, or may have a diffractive optical element such as the diffractive optical elements illustrated in FIG. 7b, or the surface may have saw-tooth structure, for example the surface may have a series of pyramidal-shaped projections or a series of pyramidal-shaped grooves.

FIG. 9c-f show alternative configurations for the speckle-reducing-optical-element; the speckle-reducing-optical-elements 190,191,192,193 shown in FIG. 9c-f have many of the same features as the speckle-reducing-optical-element 90 shown in FIG. 9a and like features are awarded the same reference numbers. In the speckle-reducing-optical-elements 190,191,192,193 the micro lens array 91 is configured such that both opposing surfaces 97a,97b of the micro lens array 91 are provided with either concave or convex contours so that the speckle-reducing-optical-elements 190,191,192,193; to achieve this the micro lens array 91 is provided with lens 92 which are shaped to have opposing surfaces which are either concave or convex.

As illustrated in FIG. 9b, during use, each of the rows 101a-c can produce pixels 8 of different lengths ‘w’,‘v’,k′ when they reflect light 11 to the entity 2. The user may select which length of pixel 8 they desire; and then can adjust the oscillation of the MEMS micro minor 105 so that light 11 is passed, from the MEMS micro mirror 105, only to the micro-lenses 91 of the one of the three rows 101a-c which produces that desired length pixel 8. The thickness ‘t’ of the pixels 8 may also be adjusted by adjusting the laser modulation speed; to increase the thickness ‘t’ of the pixels 8 the laser modulation speed should be decreased, and to decrease the thickness of the pixels the laser modulation speed should be increased.

FIG. 9g illustrates another speckle-reducing-optical-element 120 which could be used in a device according to any of the embodiments of the present invention. As shown the speckle-reducing-optical-element 120 comprises a diffractive optical element 110, instead of a micro-lens array 91. A beam-splitting layer 95 and reflective layer 96 are provided on opposing surfaces of the diffractive optical element 110. It will be understood that a diffractive optical element 110 is a structure which is shaped to have a plurality of projections 111, the plurality of projections 111 having two or more different heights ‘h’; the projections 111 are shaped and arranged so that the diffractive optical element 110 diffracts light it receives to a shaped that light so that the light can form line(s), dot(s) or any predefined shaped pixel 8 when projected onto the entity. In order to form line shaped pixels, the projections 111 of the diffractive optical element 110 should be arranged to extend perpendicular to the line shaped pixels to be created. The diffraction of light from each of the projections 111 of the diffractive optical element 110, follows the law of diffraction W*sin(a)=n*l, where ‘a’ is the diffraction angle, ‘l’ is the wavelength of the light, ‘n’ is an integer and ‘W’ is the width of the projection 111. The smaller ‘W’ is the larger is the diffraction angle. To obtain visible diffraction (large enough ‘a’), the size of the projection 111 shall have the width (W) of 0.1 to 1000 times the wavelength of the light which is incident on the projections 111 of the diffractive optical element 110. The width “W” of each projection 111 is preferably in the order of magnitude of 0.1 to 1000 times the wavelength of the light to have visible diffraction of light. Light incident on each of the projections 111 is diffracted by that respective projection 111 in a direction which is perpendicular to the longest side of the projection 111. As a result a line shaped pixel is generated from the light beams diffracted by the diffractive optical element 110.

In another variation of the speckle-reducing-optical-element 90 shown in FIG. 9a, the speckle-reducing-optical-element may alternatively comprise a diffractive grating instead of a micro-lens array 91, and the beam-splitting layer 95 and reflective layer 96 are provided on opposing surfaces of the diffractive grating. In a further variation the speckle-reducing-optical-element 90 may alternatively comprise a pyramid array instead of a micro-lens array 91, and the beam-splitting layer 95 and reflective layer 96 are provided on opposing surfaces of the pyramid array.

Various modifications and variations to the described embodiments of the invention will be apparent to those skilled in the art without departing from the scope of the invention as defined in the appended claims. Although the invention has been described in connection with specific preferred embodiments, it should be understood that the invention as claimed should not be unduly limited to such specific embodiment.

1. A method for detecting the positioning of an entity, comprising the steps of:
(a) using a projector, which comprises a laser and a MEMS micro mirror arranged to receive light from the laser and which can oscillate about at least one oscillation axis, to project light towards the entity to project an image which is composed of pixels onto the entity, wherein the image has a first density of pixels;
(b) changing the density of pixels in the projected image;
(c) sensing at least a portion of light of the projected image which is reflected from the entity; and
(d) using the sensed portion of light to determine the position of the entity.

2. A method according to claim 1, wherein the method further comprises repeating steps (c) and (d) a plurality of times to obtain a plurality of determined positions of the entity, and using the plurality of determined positions to determine movement of the entity.

3. A method according to claim 1, wherein the method further comprises the steps of:
identifying an area of interest within the image; and wherein the step of changing the density of pixels in the projected image comprises increasing the density of pixels in the area of interest only.

4. A method according to claim 3, wherein the step of changing the density of pixels in the projected image comprises, decreasing the density of pixels outside of the area of interest.

5. A method according to claim 1, wherein the step of changing the density of pixels in the projected image comprises, at least one of, changing the laser modulation speed; changing the speed at which the MEMS micro minor oscillates about its at least one oscillation axis; and/or changing the amplitude of oscillation of the MEMS micro mirror about one or more of its at least one oscillation axis.

6. A method according to claim 1, wherein the step of using a projector to project light towards the entity to project an image which is composed of pixels onto the entity, comprises, emitting light from the laser; passing the light through a first lens; passing the light through a second lens; and directing the light towards the entity using the MEMS micro mirror, wherein the MEMS micro minor is configured to oscillate about a single oscillation axis only.

7. A method according to claim 1, wherein the step of using a projector to project light towards the entity to project an image which is composed of pixels onto the entity, comprises, emitting light from the laser; passing the light through a collimating lens; passing the light through a first lens; and directing the light towards the entity using the MEMS micro minor, wherein the collimating lens is configured to collimate the light beam in a first and second plane, wherein the first and second planes are orthogonal; and the first lens is configured to focus light along a third plane which is orthogonal to the first and second planes and wherein the MEMS micro mirror is configured to oscillate about a single oscillation axis only.

8. A method according to claim 1, wherein the projector further comprises a diffractive optical element, and wherein the step of using a projector to project light towards the entity to project an image which is composed of pixels onto the entity, comprises reflecting the light using a diffractive optical element, wherein the diffractive optical element is integral to a reflective of the MEMS micro minor and wherein the step of using a projector to project light towards the entity to project an image which is composed of pixels onto the entity, comprises emitting light from the laser; passing the light through a first collimating lens which is configured to collimate light in two orthogonal planes; and directing the collimated light towards the entity using a diffractive optical element which is integral to a reflective of the MEMS micro minor.

9. A device for detecting the positioning of an entity, comprising:
a projector, which comprises a laser and a MEMS micro minor arranged to receive light from the laser and which can oscillate about at least one oscillation axis, to project light towards the entity to project an image which is composed of pixels onto the entity, wherein the image has a first density of pixels;
a controller which is configured to adjust the device so as to change the density of pixels in the projected image; and
a sensor which is configured to sense at least a portion of the light of the projected image which is reflected from the entity and configured to use the sensed portion of the light to determine the position of the entity.

10. A device according to claim 9, wherein the sensor is further configured to determine movement of the entity using a plurality of determined positions of the entity.

11. A device according to claim 9, wherein the controller is further configured to identify an area of interest within the image; and to increase the density of pixels in the area of interest only.

12. A device according to claim 11, wherein the controller is configured to decrease the density of pixels outside of the area of interest.

13. A device according to claim 9, wherein the controller is configured to change at least one or, the laser modulation speed; the speed at which the MEMS micro minor oscillates about one or more of its at least one oscillation axis; the amplitude of oscillation of the MEMS micro minor about one or more of its at least one oscillation axis, so as to change the density of pixels in the projected image.

14. A device according to claim 9, wherein the projector further comprises a first lens which is configured to collimate light along a first plane, and a second lens which is configured to focus light along a second axis, wherein the first and second planes are each orthogonal to one another, and wherein the first and second lenses are located in an optical path between the laser and the MEMS micro minor.

15. A device according to claim 9, wherein the projector further comprises a collimating lens which is configured to collimate light along a first and second plane, wherein the first and second planes are orthogonal, and a first lens which is configured to focus light along a third plane, wherein the third plane is orthogonal to each of the first and second planes, wherein the collimating lens is arranged to receive light form the laser, and wherein the MEMS micro minor is arranged to receive light from collimating lens, and wherein the first lens is arranged to receive light which is reflected by the MEMS micro minor.

16. A device according to claim 9 wherein the projector further comprises a diffractive optical element which is integral to a reflective surface of the MEMS micro mirror.

17. A device according to claim 9, wherein the projector further comprises a speckle-reducing-optical-element which is arranged to receive light which is reflected from the MEMS micro minor and to direct light towards the entity, wherein the speckle-reducing-optical-element comprises one of, a micro-lens array comprising a reflective layer and a beam-splitting layer; a diffractive optical element comprising a reflective layer and a beam-splitting layer; or a diffractive grating comprising a reflective layer and a beam-splitting layer.

18. A method of measuring distance, comprising the steps of:
(a) using a projector to project light towards an entity to project an image which composed of pixels onto the entity, wherein the image has a first density of pixels;
(b) changing the density of pixels in the projected image;
(c) sensing at least a portion of light of the projected image which is reflected from the entity; and
(d) using the sensed portion of the light to determine the distance of the entity from the projector.

19. A device for measuring distance, comprising:
a projector, which comprises a laser and a MEMS micro minor arranged to receive light from the laser and which can oscillate about at least one oscillation axis, to project light towards the entity to project an image which is composed of pixels onto the entity, wherein the image has a first density of pixels;
a controller which is configured to change the density of pixels in the projected image; and
a sensor which is configured to sense at least a portion of the light of the projected image which is reflected from the entity and configured to use the sensed portion of the light to determine the distance of the entity away from the projector.
